(19) United States
(12) Patent Application Publication
(10) Pub. No.: US 2020/0261532 A1
(43) Pub. Date: Aug. 20, 2020

(54) Chinese Medicine Composition for Treating Gastric Reflux

(71) Applicant: YinMing Li, Hong Kong (CN)

(72) Inventor: YinMing Li, Hong Kong (CN)

(21) Appl. No.: 16/649,742

(22) PCT Filed: Jan. 17, 2018
(86) PCT No.: PCT/CN2018/073014

§ 371 Date: Mar. 23, 2020

(30) Foreign Application Priority Data
Sep. 25, 2017 (HK) 17109726.3
Dec. 7, 2017 (CN) 201711288803.1

(51) Int. Cl. A61K 36/9068; A61K 36/9066; A61K 36/236; A61K 36/288; A61K 36/744; A61K 36/258; A61K 36/899; A61K 36/484; A61K 36/73; A61K 36/752; A61K 36/725; A61K 35/618

(52) U.S. Cl. CPC A61K 36/9068; A61K 36/9066; A61K 36/236; A61K 36/288; A61K 36/744; A61K 35/618; A61K 36/899; A61K 36/484; A61K 36/73; A61K 36/752; A61K 36/725; A61K 36/258

(57) ABSTRACT

A Chinese medicine composition for treating gastric reflux, comprising the following raw materials in parts by weight: 2-15 g of turmeric root tuber, 2-10 g of Gardenia jasminoides, 2-15 g of bamboo shavings, 2-10 g of Panax notoginseng, 3-12 g of ginger, 10-30 g of nacre, 3-12 g of fermented soybean, 5-20 g of cuttlebone, 3-12 g of honey-fried licorice root, 5-25 g of loquat leaf, 3-15 g of dried tangerine peel, 5-25 g of dandelion, and 5-25 g of jujube. The preparation method comprises the following steps: collecting and washing the raw materials; boiling the raw materials in water; combining, concentrating and cooling filtrates; adding ethanol so that the alcohol content is 20-80%; recovering ethanol; concentrating a supernatant into a thick paste; and saving the thick paste for later use. The thick paste is dried and prepared into corresponding formulations, including pills, tablets, powders, granules or soup bags.

### TECHNICAL FIELD

The invention belongs to the technical field of traditional Chinese medicine, and particularly relates to a Chinese medicine composition for treating gastric reflux and a preparation method thereof.

### BACKGROUND

Before food enters the stomach, there is a cartilaginous valve called the lower esophageal sphincter. Normally when swallow, the valve opens, and as the food passes through the esophagus into the stomach, the valve closes again. However, when the valve does not open and close properly, food and gastric acid in the stomach may flow back into the esophagus. Gastric reflux is one of the main symptoms of gastro-esophageal reflux disease. Since the lower esophageal sphincter could not function properly, gastric acid flows from the stomach back into the esophagus. The main causes of gastric reflux are: (1) poor living habits, for example eating at night, when food has not been fully digested, people go to bed with a full stomach, which may cause gastroptosis easily and relax the lower sphincter, causing gastric acid to flow back into the esophagus; (2) the pants are too tight and tend to push gastric acid back into the esophagus; (3) obesity, there is too much abdominal fat to cause a certain pressure to the stomach; (4) the habits of smoking and drinking can lead to excessive secretion of gastric acid; (5) for pregnant women, during late pregnancy, the baby will press on the stomach and cause gastric reflux; (6) life stress, due to the big work stress, intense rhythm and irregular eating hours of modern life, gastric reflux and stomach dysfunction may be caused. Common symptoms of gastric reflux include persistent cough, persistent sore throat, dysphagia; a burning sensation in the chest, up to the throat; a sour or bitter taste in the mouth; stomachache, hiccup, gaseous distention, toothache and asthma.

If the symptoms of gastric reflux are not serious, they can be alleviated by self-adjustment. For example, life styles are adjusted, including avoiding eating and also trying not to drink water three to four hours before bedtime; avoiding lying down after meals; eating more frequent, smaller meals to reduce the pressure on the stomach; avoiding wearing tight clothing; sleeping with head elevated about 10 centimeters so as to alleviate gastric reflux at night; cutting down on chocolates, coffee, tea, greasy and fried food, onion, garlic and peppermint food; controlling nausea symptoms with medication, taking medicine half an hour before meals to help digest food.

Gastric reflux is often mistaken for a heart problem, which needs a series of heart tests to confirm the cause of gastric reflux. Furthermore, sore throat, hoarseness, persistent cough and asthma can all be caused by gastric reflux. If the afore-mentioned symptoms appear for a long time and can not be cured after multiple treatments, it's probably gastric reflux. Gastric acid can flow back into the trachea, causing cough. Gastric acid may also irritate the sympathetic nerves of the esophagus, directly affecting tracheal contraction and causing asthma symptoms. Gastric reflux also causes direct damages to the esophagus. If untreated for a long time, gastric reflux may lead to esophagitis, esophageal ulcer, and even a precancerous lesion of Barrett's esophageal cancer, increasing the risk of cancer. The degree of gastric reflux symptoms is not proportional to the extent of damages to the esophagus. Some patients have significant gastric reflux symptoms, but there are minor damages to the esophagus; on the contrary, some patients have no obvious symptoms, but the esophagus has ulcerated badly.

There are drugs for gastric reflux on the market, but they can not recover the elasticity of the sphincter, also can not treat asthma, runny nose, breath disturbance, toothache, sore throat and cough caused by gastric reflux, and pregnant women should also take them with caution. However, similar western medicines treat the symptoms, not the disease, with a narrow therapeutic range. Each western medicine is only for the stomach, only for shortness of breath, only for sore throat, or only for cough, while can not treat other symptoms caused by gastric reflux at the same time. Furthermore, there are also many risks associated with surgery, such as dysphagia. If a new technology of adding electric ring is used, the test should be done every six months, and the electric ring also needs to be implanted into the body for a long time. At present, there is no drugs that can treat gastric reflux effectively except surgery.

### SUMMARY

For solving the above problems, the present invention aims to provide a Chinese medicine composition for treating gastric reflux by recovering the elasticity of the sphincter.

To achieve the above purpose, the present invention employs the following technical scheme:

A Chinese medicine composition for treating gastric reflux, comprising the following raw materials in parts by weight:
2-15 g of turmeric root tuber, 2-10 g of Gardenia jasminoides, 2-15 g of bamboo shavings, 2-10 g of Panax notoginseng, 3-12 g of ginger, 10-30 g of nacre, 3-12 g of fermented soybean, 5-20 g of cuttlebone, 3-12 g of licorice root (honey-fried), 5-25 g of loquat leaf, 3-15 g of dried tangerine peel, 5-25 g of dandelion, and 5-25 g of jujube.

Preferably, the Chinese medicine composition for treating gastric reflux comprises the following raw materials in parts by weight:
8-12 g of turmeric root tuber, 4-7 g of Gardenia jasminoides, 8-12 g of bamboo shavings, 2-5 g of Panax notoginseng, 3-5 g of ginger, 20-27 g of nacre, 5-8 g of fermented soybean, 12-17 g of cuttlebone, 5-8 g of licorice root (honey-fried), 10-20 g of loquat leaf, 7-10 g of dried tangerine peel, 12-17 g of dandelion, and 12-17 g of jujube.

Further preferably, the Chinese medicine composition for treating gastric reflux comprises the following raw materials in parts by weight: 10 g of turmeric root tuber, 5 g of Gardenia jasminoides, 10 g of bamboo shavings (ginger), 3 g of Panax notoginseng, 2 pieces of ginger, 25 g of nacre, 6 g of fermented soybean, 15 g of cuttlebone, 6 g of licorice root (honey-fried), 15 g of loquat leaf, 9 g of dried tangerine peel, 15 g of dandelion, and 15 g of jujube.

Preferably, the bamboo shavings is ginger bamboo shavings which is a medicine made of bamboo shavings. The processing method for ginger bamboo shavings includes any one of frying, steaming or baking.

Specific preparation methods of ginger bamboo shavings are as follows respectively:
Frying: taking bamboo shavings to remove impurities and hard skin, they are cut into segments or kneaded into a lump, mixed well with ginger juice to make it moist, placed in a frying container after the ginger juice has been absorbed, heated with a slow fire, just like making a pancake, burning both sides until slightly yellow, taken out and cooled down. The weight ratio between bamboo shavings and ginger is 10:1-2. Preferably, the weight ratio between bamboo shavings and ginger is 10:1.

Steaming: taking 100 kg of bamboo shavings medicinal raw materials to remove impurities and hard skin, they are cut into segments or kneaded into small lumps; taking 10-20 kg of ginger, it is mashed and added with 2-3 kg of water to slag juice. The ginger juice is sprayed on the bamboo shavings, then mixed well and let sit for 5 minutes. After the ginger juice has been absorbed completely by bamboo shavings, they are placed in a pot and steamed for 5 to 10 minutes, taken out while hot and dried in air. Preferably, the weight ratio between bamboo shavings and ginger is 10:1.

Baking: taking 50 kg of clean bamboo shavings, it is mixed with ginger juice taken from 12.5-15 kg of ginger and let sit for a while. After the ginger juice has been absorbed completely, they are baked at 80° C. for about 140 minutes. Then the temperature is raised up to 150° C., they are baked continually for 30 minutes to slightly yellow. Preferably, the weight ratio between bamboo shavings and ginger is 4:1.

Preferably, the licorice root is a honey-fried licorice root. The honey-fried licorice root is a leguminous plant, which is a licorice root baked with honey. It is cut into round or oval like sections, the surface is reddish-brown or greyish-brown with slightly glossy, and the section surface is yellow to dark yellow, with distinct cambium rings and radiating rays. The soup prepared with honey-fried licorice root—honey-fried licorice root soup has therapeutic effects of benefiting Qi and nourishing Yin, activating Yang and restoring pulse.

The processing method is as follows: the honey is firstly refined into medium honey in a pot, then stirred evenly with licorice root slices over a slow fire for 3 to 5 minutes, with a weight ratio between licorice root and honey of 4-5:1-2. After taken out from the pot, they are baked in a curing barn or a baking oven at 60-70° C. until hands don't stick, then taken out and cooled in air.

Further preferably, the weight ratio between licorice root and honey is 4:1, the resulting honey-fried licorice root is not easy to burn, with a good quality.

Preferably, the dosage forms of the Chinese medicine composition for treating gastric reflux are formulated into pills, tablets, powders, granules or soup bags.

Further preferably, the Chinese medicine composition for treating gastric reflux added with pharmaceutically acceptable adjuvant are formulated into pills, tablets, powders, granules or soup bags.

The Chinese medicine composition for treating gastric reflux of the invention will be prepared in the best way by a joint venture registered in Hong Kong, the preparation method of which can be easily available to persons skilled in the art based on the prior art.

Preferably, the preparation method of the Chinese medicine composition for treating gastric reflux of the invention comprises the following steps:
(1) collecting and washing the raw materials, boiling the raw materials in water; combining, concentrating and cooling filtrates; adding ethanol so that the alcohol content is 20-80%; recovering ethanol; concentrating a supernatant into a thick paste; and saving the thick paste for later use;
(2) drying the thick paste, then adding pharmaceutically acceptable adjuvants to formulate into pills, tablets, powders, granules or soup bags.

Various Chinese herbal medicines used in the invention have the following tastes and efficacies:

Turmeric root tuber is the tuberous root of curcuma aromatica salisb, cold in nature, acrid or bitter in taste, distribute to liver, heart or lung channel, having the effects of activating blood circulation and relieving pain, promoting Qi circulation and relieving depression, clearing heart and cooling blood, and increasing choleresis and relieving jaundice, which is mainly used for the treatment of pain in chest and hypochondrium, chest stuffiness and pains, amenorrhea and algomenorrhea, breast distending pain, fever and coma, epilepsy and delirium, blood heat and epistaxis, jaundice and dark urine, and the like. Modern studies have indicated that turmeric root tuber contains volatile oils (camphene, camphor, sesquiterpene, etc.), curcumin and turmerone, further contains starches, polysaccharides, fatty oils, rubbers, phellandrene and the like; which has the effects of protecting liver cells, promoting liver cell regeneration, degreasing and inhibiting liver cell fibrosis, thus defensing against toxic hepatopathies.

The fruits of Gardenia jasminoides are traditional Chinese medicine, belonging to the first batch of medicine and food resources issued by the Ministry of Health, which is cold in nature, bitter in taste, non-toxic, distribute to heart, lung, stomach or Tri-jiao channel, having the effects of liver protection, choleresis increasing, depressurization, sedation, hemostasis, and detumescence. It is often used for the treatment of jaundice hepatitis, sprain and contusion, hypertension, and diabetes in the clinic of traditional Chinese medicine. It is found in modern pharmacological studies that Gardenia jasminoides can promote pancreatic secretion. For pancreatitis, Gardenia jasminoides has pharmacological effects of strengthening the body's resistance to disease, improving the functions of liver and gastrointestinal system, and alleviating pancreatitis.

Bamboo shavings is the dry intermediate layer of the stems of Bambusa tuldoides Munro, Sinocalamus beecheyanus, or Phyllostachys glauca belonging to gramineous plants. It is slightly cold in nature, sweet in taste, distribute to liver, stomach or gallbladder channel, having the effects of clearing away heat and dispersing phlegm, relieving restlessness and preventing vomiting, which is used for the treatment of heat cough with phlegm, gallbladder fire with phlegm, palpitate with fear and restless, insomnia, stroke with phlegm, stiff tongue impeding speech, vomiting due to heat in stomach, pernicious vomiting with pregnancy, and fetal irritability. Ginger bamboo shavings specializes in dispersing phlegm and preventing vomiting.

Panax notoginseng, also known as pseudo-ginseng, is a plant of Araliaceae, Umbellales, warm in nature; sweet and slightly bitter in taste; distribute to liver, stomach, heart, lung or large intestine channel; having the effects of removing stasis and arresting bleeding, activating blood circulation and relieving pain, which is used for the treatment of hemoptysis, hematemesis, epistaxis, hematochezia, metrorrhagia and metrostaxis, traumatic bleeding, sharp pain in the chest and abdomen, swelling and pain from bruises.

Ginger refers to the tuberous root of Zingiber plants, slightly warm in nature, acrid or sweet in taste; distribute to lung, spleen or stomach channel; its specific “gingerol” can irritate gastrointestinal mucosa, causing hyperaemia of the gastrointestinal tract and enhancing the digestive ability, thus effectively treating abdominal distension, stomachache, diarrhea, and emesis caused by eating too much cold food. Modern pharmacological studies have indicated that ginger is an effective drug for treating hydrochloric acid-ethanol ulcer, its active ingredient is zingiberene, which has the effect of protecting gastric mucosa cells. In aromatic stomachic crude drugs, especially zingiberaceae plants contain terpenoids essential oil components such as zingiberene, which is one of the active ingredients of stomachic crude drugs.

Nacre is the shell of Hyriopsis cumingii, Cristaria plicata of Unionidae or Pteria martensii Dunker of Pteriidae, cold in nature; salty in taste; distribute to heart or liver channel; having the effects of suppressing hyperactive liver and subsiding Yang, tranquilizing the mind and arresting convulsion, clearing liver and improving vision; which is mainly used for the treatment of dizzy, tinnitus, palpitation, insomnia, insane, epilepsy, hematemesis, epistaxis, women's metrorrhagia. Modern pharmacological studies have indicated that nacre contains a large amount of calcium carbonate, and oral administration of this powder has the functions of neutralizing gastric acid and astriction. The pear layer powder is often used for the treatment of hyperchlorhydria and ulcer diseases.

Fermented soybean is the fermented product of soybean mature seeds of legumes, which is cold in nature; acrid, sweet, or slightly bitter in taste; having the effects of relieving exterior syndromes, relieving restlessness, venting depression, and detoxification; which is used for the treatment of typhoid fever, chills and fever, headache, dysthesia, and chest distress. Modern pharmacological studies have indicated that fermented soybean has the effects of weak sweating, invigorating stomach and promoting digestion.

Licorice root is a nourishing herbal medicine, the medicinal parts are the root and the rhizome. Licorice root is sweet in taste, neutral in nature and distribute to heart, spleen, lung or stomach channel. It is cold when raw, and can be used for heat-clearing and detoxicating, relaxing spasm and relieving pain; while it is warm when fried, and can be used for dispersing exterior cold, and tonifying middle-Jiao and Qi. Furthermore, licorice root is also good at coordinating the nature of drugs and detoxicating all medicines.

Cuttlebone is the dry inner shell of Sepiella or Sepia esculenta of Sepiidae, warm in nature; salty or puckery in taste; distribute to spleen or kidney channel; having the effects of astringency and hemostasis, arresting seminal emission and leukorrhea, stopping acid regurgitation and relieving pain, astringing dampness and healing sore, which is often used for the treatment of hematemesis, epistaxis, metrorrhagia and metrostaxis, hematochezia, spermatorrhea and night emission, leukorrhea with reddish discharge, stomachache and acid regurgitation; and used for external treatment of bleeding injuries, eczema and wet sores, and unhealing ulcers.

Loquat leaf is the leaf of loquat of Rosaceae with delicate fragrance, slightly bitter in taste, and slightly cold in nature, having the effects of removing heat from the lung and arresting cough, harmonizing stomach and diuresis, and quenching thirst, which can be used for the treatment of cough with phlegm due to heat in lung, hemoptysis, epistaxis, and vomiting due to heat in stomach.

Dried tangerine peel is the dried ripe peel of tangerine of Rutaceae and its cultivated varieties. This medicinal material is divided into “dried tangerine peel” and “citrus chachiensis hortorum”. Ripe fruits are picked and peeled, then the peels are dried in the sun or freeze-dried. The dried tangerine peel is warm in nature, acrid or bitter in taste; distribute to spleen, stomach, or lung channel; having the effects of regulating Qi and strengthening spleen, regulating middle energizer, eliminating dampness, and dissipating phlegm, which is mainly used for the treatment of abdominal fullness and distention or pain and dyspepsia caused by Qi stagnation of spleen and stomach, chest distress and abdominal distension, poor appetite and loose stools caused by turbid dampness, cough and asthma caused by accumulation of phlegm-dampness in the lung, chest fullness and distention, vomiting and diarrhea with eating less, cough with abundant phlegm.

Dandelion is a perennial herb of Asteraceae, neutral in nature, sweet or slightly bitter in taste, having the effects of clearing heat and removing toxicity, reducing swelling and eliminating stagnation, and promoting lactation, which is very effective in the treatment of mastitis. Furthermore, dandelion also has the effects of diuresis, mitigating diarrhea, relieving jaundice, and increasing choleresis. Modern pharmacological studies have indicated that dandelion has the function of resisting gastric ulcer, and has protective effects on gastric mucous membrane injuries.

Jujube is very high in vitamins and has the reputation of “the natural vitamin pill”. It is slightly warm in nature and sweet in taste, having the effects of invigorating spleen and replenishing Qi, and nourishing the blood and tranquilizing the mind.

The present invention is used to treat gastric reflux caused by stagnation of liver Qi, insufficiency of spleen-Qi and stomach-Qi, pathogenic dampness encumbering the spleen, dysfunction of spleen in transportation, transportation weakness, sphincter relaxation, stomachache and acid regurgitation, insufficient Qi and blood biochemistry, stomach Qi rising, and stagnation of vital energy.

Effusion leads to stagnation of liver Qi, and liver disease tends to spread to the spleen. The spleen and stomach are the root of after-birth and the source of Qi and blood. The weakness of spleen and stomach will lead to insufficient Qi and blood, dysfunction of the spleen in transport, anepithymia, endogenous turbid dampness, stagnation of Qi activity, ascending lucidity and descending turbidity disorder. Improper diet would cause epigastric abdominal stuffy and full, hyperacidity, esophageal reflux, rustiness, shortness of breath, lassitude, asthma and palpitation. Treatment should focus on soothing the liver and resolving depression, tonifying middle-Jiao and Qi, relieving Qi stagnancy in stomach, warming middle and resolving dampness, eliminating dampness and invigorating spleen, descending of Qi.

Ginger bamboo shavings is used in combination with dried tangerine peel, licorice root, ginger for clearing up phlegm and resolving depression, further used with jujube for clearing heat and stopping vomiting to treat hygropyretic vomiting, stomach Qi rising, nausea and vomiting. They also have been confirmed to be useful for the treatment of boredom restless, light-headedness, nausea and vomiting caused by gallbladder deficiency phlegm-heat depression, cooperating with the effects of dissipating phlegm, tranquilizing the mind, and preventing vomiting. Panax notoginseng is capable of removing stasis and arresting bleeding, reducing swelling and relieving pain. Cuttlebone is capable of astringency and hemostasis, stopping acid regurgitation and relieving pain. Nacre is used in combination with honey-fried licorice root to treat liver Yang rising, dizziness, headache, giddiness, tinnitus, and cheek dryness-heat. Turmeric root tuber is used in combination with Gardenia jasminoides to achieve the effects of clearing heat and cooling blood, and activating blood circulation. Fermented soybean is used in combination with Gardenia jasminoides and licorice root to treat pathogenic heat depression in chest, annoyance, insomnia caused by heat vexation. Loquat leaf is fried in combination with ginger and used together with jujube to treat hoarseness. Dandelion distributes to liver or stomach channel, having the effects of clearing heat and removing toxicity, diuresis and anti-inflammation, being effective on various inflammatory diseases, with the effects of clearing liver and increasing choleresis especially preferrable, and thus being capable of symptomatically mitigating buccal inflammation, halitosis, and gastritis caused by smoking.

In the present invention, a variety of drugs are combined to enable a synergistic effect to cure gastric reflux and treat esophagitis, esophageal reflux and laryngopharyngitis caused by gastric reflux, and further recover the elasticity of the sphincter, reduce gastric gases, condition and strengthen gastric mucous membrane, avoiding the risks of long-term consumption of palliative drugs and surgery, as well as the trouble of nursing and sequelae after operation, thus effectively treating asthma, runny nose, breath disturbance, toothache, sore throat, rustiness and cough caused by gastric reflux. Meanwhile, pregnant women can also take it, but should consult a physician for advice first due to personal physical difference.

Usage and dosage: The composition of the invention is taken 1 to 2 times a day, with 7 days for a course of treatment. For patients with mild symptoms, 1 to 2 courses of treatment is generally sufficient to cure. While for serious patients, continuous use for 1 to 3 months is recommended. Pregnant women should consult a physician for advice first before taking the medicine.

Of particular note is that for gastric reflux patients with Helicobacter pylori as the pathogenic cause, it is necessary to cure the bacteria before taking this medicine.

Beneficial effects of the invention:
1. The risks of long-term consumption of palliative drugs and surgery can be avoided by taking the medicine provided in the invention, and after operation, the pharmaceutical preparations provided by the invention are in various forms to meet the needs of different patients;
2. The trouble of nursing and sequelae can be also avoided, and asthma, runny nose, breath disturbance, toothache, sore throat, rustiness and cough caused by gastric reflux can be effectively treated;
3. The medicine of the invention can cure gastric reflux and treat esophagitis, esophageal reflux and laryngopharyngitis caused by gastric reflux, can also recover the elasticity of the sphincter, reduce gastric gases, condition and strengthen gastric mucous membrane;
4. The medicine provided by the invention also avoids the trouble of decoction and the problems of excessive levels of heavy metals and microorganisms in herbal raw materials, and it is also easy to store and carry out.

### DESCRIPTION OF THE EMBODIMENTS

The invention will be further illustrated below in detail in conjunction with the following embodiments.

### Embodiment 1

Collecting 2 g of turmeric root tuber, 2 g of Gardenia jasminoides, 2 g of ginger bamboo shavings, 2 g of Panax notoginseng, 2 pieces of ginger, 10 g of nacre, 3 g of fermented soybean, 5 g of cuttlebone, 3 g of honey-fried licorice root, 5 g of loquat leaf, 3 g of dried tangerine peel, 5 g of dandelion, and 5 g of jujube and washing, boiling the raw materials in water; combining, concentrating and cooling filtrates, adding ethanol so that the alcohol content is 20-80%, recovering ethanol, concentrating a supernatant into a thick paste; and saving the thick paste for later use. Into the thick paste is added β-cyclodextrin, heated to dissolve, stirred, filtered, and spray dried at an inlet hot air temperature of 180° C. and an outlet air temperature of 80° C. Flavouring agent is added into the spray-dried powders and blended evenly. The materials are processed by dry granulation at 40° C., integrated into granules and packaged into bags, to obtain the granules of the invention for treating gastric reflux by recovering the elasticity of the sphincter.

### Embodiment 2

Collecting 15 g of turmeric root tuber, 10 g of Gardenia jasminoides, 15 g of ginger bamboo shavings, 10 g of Panax notoginseng, 2 pieces of ginger, 30 g of nacre, 12 g of fermented soybean, 20 g of cuttlebone, 12 g of honey-fried licorice root, 25 g of loquat leaf, 15 g of dried tangerine peel, 25 g of dandelion, and 25 g of jujube and washing, boiling the raw materials in water; combining, concentrating and cooling filtrates, adding ethanol so that the alcohol content is 20-80%, recovering ethanol, concentrating a supernatant into a thick paste; and saving the thick paste for later use. Into the thick paste is added β-cyclodextrin, heated to dissolve, stirred, filtered, and spray dried at an inlet hot air temperature of 180° C. and an outlet air temperature of 80° C. Flavouring agent is added into the spray-dried powders and blended evenly. The materials are processed by dry granulation at 40° C., integrated into granules and packaged into bags, to obtain the granules of the invention for treating gastric reflux by recovering the elasticity of the sphincter.

### Embodiment 3

Collecting 15 g of turmeric root tuber, 8 g of Gardenia jasminoides, 10 g of ginger bamboo shavings, 10 g of Panax notoginseng, 2 pieces of ginger, 20 g of nacre, 12 g of fermented soybean, 10 g of cuttlebone, 8 g of honey-fried licorice root, 20 g of loquat leaf, 15 g of dried tangerine peel, 20 g of dandelion, and 20 g of jujube and washing, boiling the raw materials in water; combining, concentrating and cooling filtrates, adding ethanol so that the alcohol content is 20-80%, recovering ethanol, concentrating a supernatant into a thick paste; and saving the thick paste for later use. Into the thick paste is added 95% ethanol twice the weight of the paste, stirred thoroughly and left stand for 24 to 48 hours at room temperature, taking the supernatant. The precipitate is washed with 65% ethanol for 1 to 2 times. The washing liquid is combined with the supernatant, and if there is any alcohol extract, it is also combined with the supernatant, filtered and clarified, then charged into a reduced pressure concentration tank or an alcohol recovery tower to recover ethanol until the medicine liquid has no alcohol taste. The medicine liquid is diluted with distilled water, heated to 100° C. for the colloid aggregation, adjusted to pH 4.5-5.0, then stored at low temperature for 24 hours so that the aggregated colloid can be precipitated, and then filtered to clarification, into which is then added a proper amount of corrective agent and preservative and left stand, then filtered and clarified, and stored at low temperature. The final purification and filtration should be done before filling, and the filtrate should be checked to be clear by a light detector, and then sealed in nutrition bottles with straight tubes and easy-open caps on a machine, with each oral liquid bottle filled with 10 ml, that is the oral liquid of the invention for treating gastric reflux by recovering the elasticity of the sphincter.

### Embodiment 4

Collecting 10 g of turmeric root tuber, 5 g of Gardenia jasminoides, 10 g or ginger bamboo shavings, 3 g of Panax notoginseng, 2 pieces of ginger, 25 g of nacre, 6 g of fermented soybean, 15 g of cuttlebone, 6 g of honey-fried licorice root, 15 g of loquat leaf, 9 g or dried tangerine peel, 15 g of dandelion, and 15 g of jujube and washing, boiling the raw materials in water; combining, concentrating and cooling filtrates, adding ethanol so that the alcohol content is 20-80%, recovering ethanol, concentrating a supernatant into a thick paste; and saving the thick paste for later use. Into the thick paste is added β-cyclodextrin, heated to dissolve, stirred, filtered, and spray dried at an inlet hot air temperature of 180° C. and an outlet air temperature of 80° C. Flavouring agent is added into the spray-dried powders and blended evenly. The materials are processed by dry granulation at 40° C., integrated into granules and packaged into bags, to obtain the granules of the invention for treating gastric reflux by recovering the elasticity of the sphincter.

1. A Chinese medicine composition for treating gastric reflux, comprising the following raw materials in parts by weight:
2-15 g of turmeric root tuber, 2-10 g of Gardenia jasminoides, 2-15 g of bamboo shavings, 2-10 g of Panax notoginseng, 3-12 g of ginger, 10-30 g of nacre, 3-12 g of fermented soybean, 5-20 g of cuttlebone, 3-12 g of licorice root, 5-25 g of loquat leaf, 3-15 g of dried tangerine peel, 5-25 g of dandelion, and 5-25 g of jujube.

2. The Chinese medicine composition according to claim 1, comprising the following raw materials in parts by weight: 8-12 g of turmeric root tuber, 4-7 g of Gardenia jasminoides, 8-12 g of bamboo shavings, 2-5 g of Panax notoginseng, 3-5 g of ginger, 20-27 g of nacre, 5-8 g of fermented soybean, 12-17 g of cuttlebone, 5-8 g of licorice root, 10-20 g of loquat leaf, 7-10 g of dried tangerine peel, 12-17 g of dandelion, and 12-17 g of jujube.

3. The Chinese medicine composition according to claim 1, comprising the following raw materials in parts by weight: 10 g of turmeric root tuber, 5 g of Gardenia jasminoides, 10 g of ginger-fried bamboo shavings, 3 g of Panax notoginseng, 2 pieces of ginger, 25 g of nacre, 6 g of fermented soybean, 15 g of cuttlebone, 6 g of honey-fried licorice root, 15 g of loquat leaf, 9 g of dried tangerine peel, 15 g of dandelion, and 15 g of jujube.

4. The Chinese medicine composition according to claim 1, wherein the bamboo shavings is ginger-fried bamboo shavings.

5. The Chinese medicine composition according to claim 4, wherein the ginger-fried bamboo shavings is made of bamboo shavings and ginger by any one process of frying, steaming or baking.

6. The Chinese medicine composition according to claim 1, wherein the licorice root is honey-fried licorice root.

7. The Chinese medicine composition according to claim 6, wherein the preparation method of the honey-fried licorice root comprises the following steps: the honey is firstly refined into medium honey in a pot, then stirred evenly with licorice root slices over a slow fire, taken out from the pot, and then baked in a curing barn or a baking oven at 60-70° C. until hands don't stick, then taken out and cooled in air.

8. The Chinese medicine composition according to claim 7, wherein the weight ratio between the licorice root and the honey is 4-5: 1-2.

9. The Chinese medicine composition according to claim 1, wherein the Chinese medicine composition is formulated into pills, tablets, powders, granules or soup bags.
